(19) United States
(12) Patent Application Publication
(10) Pub. No.: US 2021/0330661 A1
(43) Pub. Date: Oct. 28, 2021

(54) Use Of Tafenoquine, Boceprevir Or Narlaprevir For Treating Infection Of SARS-COV-2 And Medical Composition Thereof

(71) Applicant: China Medical University, Taichung City (TW)

(72) Inventors: Mien-Chie Hung, Taichung City (TW); Yeh Chen, Taichung City (TW); Wen-Hao Yang, Yilan County (TW); Chia-Shin Yang, Tuku Township (TW); Yu-Lin Hung, Taichung City (TW); Yu-Quan Wang, New Taipei City (TW); Yi-Zhen Chou, Taichung City (TW); Mei-Hui Hou, Taibao City (TW); Chia-Ling Tsai, Taichung City (TW); Bao-Yue Huang, Tainan City (TW); Chian-Fang Hung, Taichung City (TW)

(73) Assignee: China Medical University, Taichung City (TW)

(21) Appl. No.: 17/237,335

(22) Filed: Apr. 22, 2021

Related U.S. Application Data
(60) Provisional application No. 63/014,222, filed on Apr. 23, 2020.
(60) Provisional application No. 63/017,783, filed on Apr. 30, 2020.

(51) Int. Cl. A61K 31/47; A61K 31/403; A61K 31/706

(52) U.S. Cl. CPC A61K 31/47; A61K 31/706; A61K 31/403

(57) ABSTRACT

The present disclosure relates to a method for preventing or inhibiting a synthesis of viral RNA of SARS-CoV-2 in a cell including contacting the cell with a sufficient concentration of tafenoquine, boceprevir or narlaprevir and a method for inhibiting an activity of a main protease (M^{pro}) of SARS-CoV-2 including contacting a SARS-CoV-2 with a sufficient concentration of tafenoquine, boceprevir or narlaprevir. A medical composition for use in a treatment of an infection of SARS-CoV-2 including a therapeutically effective amount of tafenoquine, boceprevir or narlaprevir is also provided.

### RELATED APPLICATIONS

This application claims priority to U.S. Provisional Application Ser. No. 63/014,222, filed Apr. 23, 2020, and claims priority to U.S. Provisional Application Ser. No. 63/017,783, filed Apr. 30, 2020, the contents of which are herein incorporated by reference.

### BACKGROUND

### Technical Field

The present disclosure relates to uses of tafenoquine, boceprevir or narlaprevir. More particularly, the present disclosure relates to uses of tafenoquine, boceprevir or narlaprevir for treating an infection of SARS-CoV-2.

### Description of Related Art

The severe acute respiratory syndrome coronavirus 2 (SARS-CoV-2) causing the current pandemic, coronavirus disease 2019 (COVID-19), has taken a huge toll on human lives and the global economy. It has since spread rapidly, infected more than thirteen million people globally, and caused more than 2.85 million deaths. Currently, there are no scientifically proven drugs to control this outbreak.

The major clinical symptoms of COVID-19 including fever, breathing difficulties, X-ray findings of infiltrative lung injury, and high C-reactive protein (CRP) as well as high pro-inflammatory cytokine levels in the blood serum. Furthermore, the infection of SARS-CoV-2 will cause atrophy of spleen and lymph nodes that ultimately weakens the immune system. Accordingly, there is an urgent requirement for both vaccines and small-molecule antiviral drugs in order to effectively control the spread of COVID-19.

Although hundreds of vaccine candidates are under pre-clinical testing or otherwise in clinical developments, still, antiviral drugs are indispensable to effectively treat infected people. Therefore, effective treatments against this disease are urgently needed.

### SUMMARY

According to one aspect of the present disclosure, a method for preventing or inhibiting a synthesis of viral RNA of SARS-CoV-2 in a cell includes contacting the cell with a sufficient concentration of tafenoquine, boceprevir or narlaprevir.

According to another aspect of the present disclosure, a method for inhibiting an activity of a main protease (M^{pro}) of SARS-CoV-2 includes contacting a SARS-CoV-2 with a sufficient concentration of tafenoquine, boceprevir or narlaprevir.

According to further another aspect of the present disclosure, a medical composition for use in a treatment of an infection of SARS-CoV-2 includes a therapeutically effective amount of tafenoquine, boceprevir or narlaprevir.

According to still another aspect of the present disclosure, a method for treating a subject suffering from COVID-19 includes administering to the subject in need of the medical composition of the aforementioned aspect.

According to more another aspect of the present disclosure, a method for treating a subject suffering from COVID-19 includes administering to the subject in need of the medical composition of the aforementioned aspect.

### BRIEF DESCRIPTION OF THE DRAWINGS

The patent or application file contains at least one drawing executed in color. Copies of this patent or patent application publication with color drawing(s) will be provided by Office upon request and payment of the necessary fee. The present disclosure can be more fully understood by reading the following detailed description of the embodiment, with reference made to the accompanying drawings as follows:

FIG. 1 shows a result of relative activity of SARS-CoV-2 M^{pro} after treating with tafenoquine or hydroxychloroquine at various concentrations.

FIG. 2A shows a result of melting curves of SARS-CoV-2 M^{pro} after treating with tafenoquine at various concentrations.

FIG. 2B shows a result of melting curves of SARS-CoV-2 M^{pro} after treating with hydroxychloroquine at various concentrations.

FIG. 3 shows a comparison of the far-UV CD signals (molar ellipticity at 222 nm) with the enzyme activity of SARS-CoV-2 M^{pro} from FRET-base assay with increasing amounts of tafenoquine.

FIG. 4 shows a result of soluble fractions of SARS-CoV-2 M^{pro} in the presence of different concentrations of tafenoquine or hydroxychloroquine.

FIG. 5A shows a result of limited proteolysis of SARS-CoV-2 M^{pro} by trypsin in the presence of different concentrations of tafenoquine.

FIG. 5B shows a result of limited proteolysis of SARS-CoV-2 M^{pro} by trypsin in the presence of different concentrations of hydroxychloroquine.

FIG. 6 shows a result of relative activity of SARS-CoV-2 M^{pro} after treating with different anti-HCV drugs.

FIG. 7 shows a result of inhibition rate of SARS-CoV-2 M^{pro} after treating with boceprevir.

FIG. 8 shows a result of isothermal titration calorimetry analysis of boceprevir binding to SARS-CoV-2 M^{pro}.

FIG. 9 shows a result of isothermal titration calorimetry analysis of narlaprevir binding to SARS-CoV-2 M^{pro}.

FIG. 10 shows a result of amount of viral RNA of nucleoprotein in virus-infected Vero E6 cells which are treated with tafenoquine.

FIG. 11 shows a result of the inhibition rate of viral RNA of nucleoprotein on Day 2 in Vero E6 cells treated with tafenoquine with full-time or post treatment.

FIG. 12 shows images of virus-infected Vero E6 cells treated with tafenoquine or DMSO with full-time or post treatment.

FIG. 13 shows a result of amount of viral RNA of nucleoprotein on Day 2 in Vero E6 cells treated with narlaprevir with pre-treatment.

FIG. 14 shows images of virus-infected Vero E6 cells treated with narlaprevir with pre-treatment.

### DETAILED DESCRIPTION

The present disclosure will be further exemplified by the following specific embodiments to facilitate utilizing and practicing the present disclosure completely by the people skilled in the art without over-interpreting and over-experimenting. However, these practical details are used to describe how to implement the materials and methods of the present disclosure and are not necessary.

I. Main Protease of SARS-CoV-2

SARS-CoV-2 is an enveloped and positive-sense single-stranded RNA virus. The genome of SARS-CoV-2 shares about 83% identity with the SARS coronavirus that emerged in 2002 and contains approximately 30,000 nucleotides that are transcribed into 11 open reading frames (ORFs), namely ORF1ab, ORF2 (spike protein), ORF3a, ORF4 (envelope protein), ORF5 (membrane protein), ORF6, ORF7a, ORF7b, ORF8, ORF9 (nucleocapsid protein) and ORF10. The ORF1ab encodes a long polypeptide chain, which is further processed by the main protease (“M^{pro}” hereafter) thereof to produce 16 nonstructural proteins that are essential for viral replication and transcription. The unique substrate specificity of SARS-CoV-2 M^{pro} and its importance in virus life cycle makes it to be an attractive therapeutic target for the treatment of COVID-19.

II. Preparation of Recombinant SARS-CoV-2 M^{pro}

The recombinant SARS-CoV-2 M^{pro} analyzed in the present disclosure is prepared according to the following steps. First, the full-length gene encoding SARS-CoV-2 M^{pro} (ORF1ab polyprotein residues 3264-3569, GenBank code: MN908947.3) with Escherichia coli codon usage is synthesized and subcloned into pSol SUMO vector using Expresso® Solubility and Expression Screening System (Lucigen). A pET16b plasmid encoding the fluorescent protein substrate of SARS-CoV-2 M^{pro} (His₁₀-mTurquoise2-TSAVLQSGFRKM-mVenus) is synthesized and constructed for fluorescence resonance energy transfer (“FRET” hereafter) based high-throughput screening assay. Each expression plasmid is transformed into E. coli BL21 (DE3) and then grown in Luria Broth medium at 37° C. until the value of OD₆₀₀ thereof reached between 0.6 and 0.8. Then, overexpression of SARS-CoV-2 M^{pro} or the fluorescent protein substrate thereof is induced by adding 20% L-rhamnose or 0.5 mM IPTG and incubated for 18 hours at 20° C. After incubating for 18 hours, the cell pellets are resuspended in a sonication buffer [50 mM Tris-HCl at pH 8.0, 500 mM NaCl, 10% glycerol, 1 mM tris(2-carboxyethyl)phosphine (TCEP), 1 mM phenylmethylsulfonyl fluoride (PMSF)] and lysed by sonication on ice. Following centrifugation at 28,000×g, 4° C. for 30 minutes, and the supernatant is loaded onto a HisTrap FF column (GE Healthcare), washed by the sonication buffer containing 10 mM imidazole, and eluted with a 20 mM-200 mM imidazole gradient in the sonication buffer. An adequate amount of TEV protease is added to remove the N-terminal SUMO fusion tag of SARS-CoV-2 M^{pro}. Both TEV protease and Hiss-SUMO fusion tag are then removed by HisTrap FF column. Finally, the SARS-CoV-2 M^{pro} and the substrate protein thereof are further purified by size-exclusion chromatography and stored in buffer containing 50 mM Tris-HCl at pH 8.0, 200 mM NaCl, 5% glycerol, and 1 mM TCEP for following analysis.

III. Assessment of the Inhibiting Efficiency to SARS-CoV-2 M^{pro}

<Assessing the Inhibiting Efficiency of Tafenoquine to SARS-CoV-2 M^{pro}>

In the present disclosure, it is shown that tafenoquine (TFQ) can induce a significant conformational change in SARS-CoV-2 M^{pro} and diminish the protease activity thereof. Accordingly, tafenoquine has potentials to be used as a drug to treat patients suffering from COVID-19 in order to save lives and reduce the damage provoked by this outbreak. The structure of tafenoquine is shown as Formula (I).

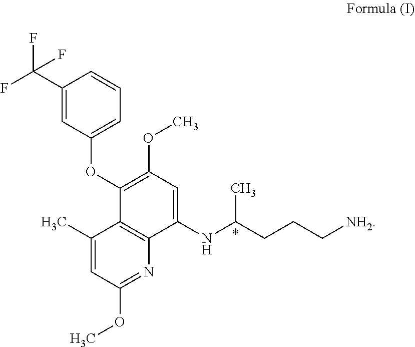 [SMILES: COc1cc(C)c2c(Oc3cccc(C(F)(F)F)c3)c(OC)cc(NC(C)CCCN)c2n1]

The inhibiting efficiency of tafenoquine to SARS-CoV-2 M^{pro} is assessed by FRET assay and differential scanning fluorimetry (“DSF” hereafter) assay.

In the experiment of FRET assay treating with tafenoquine, 4 μM SARS-CoV-2 M^{pro} is mixed with an assay buffer (20 mM Tris-HCl at pH 7.8, 20 mM NaCl) and then pre-incubated with tafenoquine for 30 minutes at room temperature in 96-well black optiplate. The reaction is initiated by adding 20 μM fluorescent protein substrate. Substrate cleavage is monitored continuously for 1 hour by detecting mTurquoise2 fluorescence (excitation: 434 nm/emission: 474 nm) using Synergy™ H1 hybrid multi-mode microplate reader (BioTek Instruments, Inc.). The first 15 minutes of the reaction is used to calculate initial velocity (V₀) by linear regression. The calculated initial velocity with tafenoquine is normalized to a DMSO control. The IC₅₀ is calculated by plotting the initial velocity against various concentrations of tafenoquine by use of a dose-response curve in Prism 8.0 software (GraphPad).

DSF assay is a powerful tool in early drug discovery with the basic principle that drugs that bind to the therapeutic protein target will stabilize it and cause a positive shift in its melting temperatures (Tm). In the experiment of DSF assay, a buffer including 25 mM Tris at pH 8.0, 150 mM NaCl, 5×SYPRO Orange dye (Sigma-Aldrich) and 8 μM SARS-CoV-2 M^{pro} in the presence of tafenoquine in each well is incubated on a CFX96 RT-PCR instrument. Fluorescence is monitored when the temperature is gradually raised from 25° C. to 90° C. in 0.3° C. increments at 12-second intervals. Melt curve data are plotted using the Boltzmann model to obtain the temperature midpoint of unfolding of the protein using Prism 8.0 software.

Furthermore, in the present experiment, hydroxychloroquine (HCQ) is used to react with SARS-CoV-2 M^{pro} so as to further confirm the inhibiting efficiency of tafenoquine to SARS-CoV-2 M^{pro}.

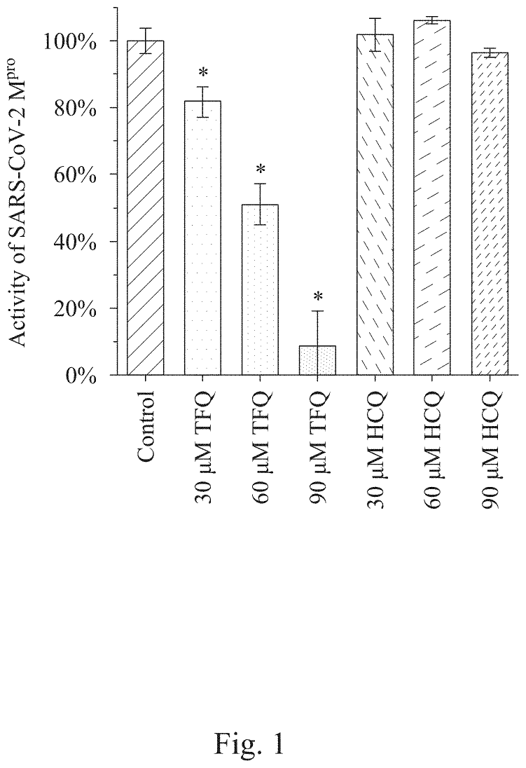

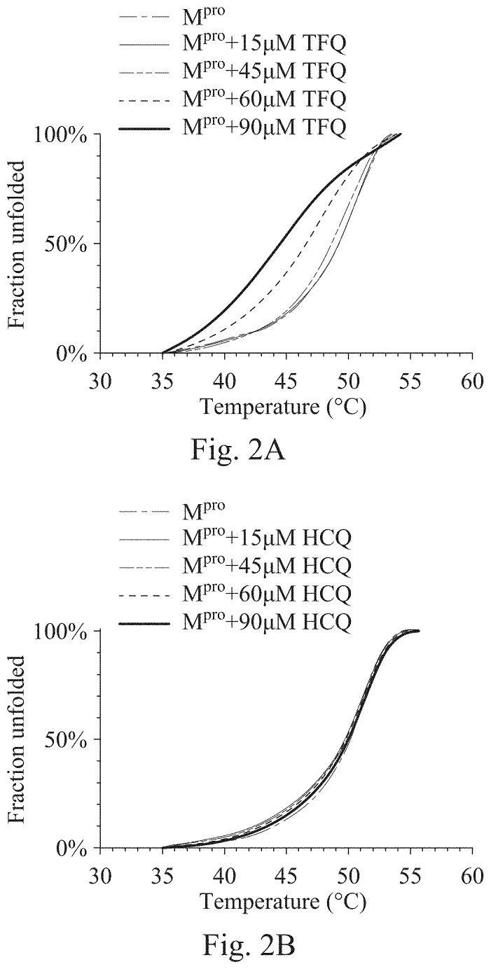

Please refer to FIGS. 1, 2A, 2B and Table 1, wherein FIG. 1 shows a result of relative activity of SARS-CoV-2 M^{pro} after treating with tafenoquine or hydroxychloroquine at various concentrations, FIG. 2A shows a result of melting curves of SARS-CoV-2 M^{pro} after treating with tafenoquine at various concentrations, FIG. 2B shows a result of melting curves of SARS-CoV-2 M^{pro} after treating with hydroxychloroquine at various concentrations, and Table 1 shows the melting temperatures of SARS-CoV-2 M^{pro} after treating with tafenoquine and hydroxychloroquine at various concentrations.

TABLE 1

| Tm (° C.) | Tafenoquine | Hydroxychloroquine |
|---|---|---|
| 15 μM | 51.0 | 51.1 |
| 45 μM | 49.9 | 51.1 |
| 60 μM | 48.3 | 51.2 |
| 90 μM | 45.6 | 51.2 |

As shown in FIG. 1, tafenoquine exhibits almost 90% inhibition against SARS-CoV-2 M^{pro} at a concentration of 90 μM whereas hydroxychloroquine does not demonstrate any significant inhibitory effects. Furthermore, as shown in Table 1, FIG. 2A and FIG. 2B, it is shown that tafenoquine causes a negative shift in the melting temperature of SARS-CoV-2 M^{pro} in a dose-dependent manner. However, hydroxychloroquine has no influence on the thermal stability of SARS-CoV-2 M^{pro}. Furthermore, in order to proof that the negative shift in the melting temperature of SARS-CoV-2 M^{pro} is not resulted by small-molecule inhibitors by disrupting their oligomeric interfaces, the analytical ultracentrifugation (AUC) assay is performed correspondingly, and the result of the analytical ultracentrifugation assay revealed identical sedimentation coefficient of SARS-CoV-2 M^{pro} at various concentrations of tafenoquine, suggesting the absence of dimer-to-monomer conversion of SARS-CoV-2 M^{pro} in the presence of tafenoquine. In other words, the binding of tafenoquine will not disrupt the dimerization interface or bind to the non-native state of SARS-CoV-2 M^{pro}.

Furthermore, the enzyme activity of SARS-CoV-2 M^{pro} from FRET-base assay with increasing amounts of tafenoquine is further compared with the far-UV circular dichroism (CD) signals (molar ellipticity at 222 nm; A222) so as to illustrate the relationship between the protein conformation and the increasing amounts of tafenoquine. Circular dichroism signals are measured using a Jasco J-815 spectropolarimeter with 0.1-cm quartz cuvettes and a 1-mm slit width. The molar ellipticity at 222 nm of all samples is recorded to analyze the protein conformational changes at different concentrations of tafenoquine (10 μM-500 μM). All spectra are corrected for buffer absorption.

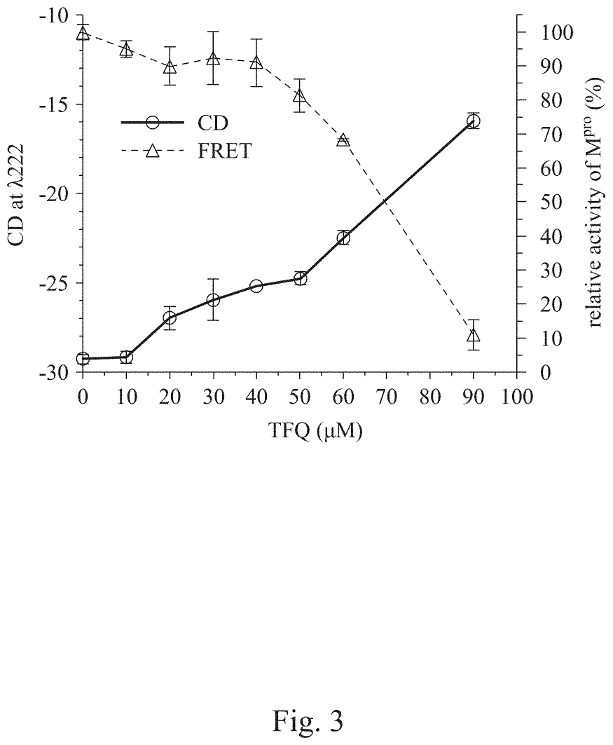

Please refer to FIG. 3, which shows a comparison of the far-UV CD signals with the enzyme activity of SARS-CoV-2 M^{pro} from FRET-base assay with increasing amounts of tafenoquine. In FIG. 3, the results are shown as a solid line (circular dichroism signals) or dashed line (enzyme activity measured by FRET) with error bars from at least two replicates. As shown in FIG. 3, a decreased α-helical content may be accompanied by reduced M^{pro} protease activity, suggesting that tafenoquine may cause a local conformational change within its binding site and then disrupts nearby α-helices and subsequently reduces the protease activity of SARS-CoV-2 M^{pro}.

Moreover, according to the aforementioned results, because the sedimentation coefficient of SARS-CoV-2 M^{pro} remains unchanged with tafenoquine, it is unlikely that tafenoquine causes unfolding of the overall structure of SARS-CoV-2 M^{pro}. In order to confirm whether the overall structure of SARS-CoV-2 M^{pro} is changed corresponding to the binding of tafenoquine or not, the solubility and stability of SARS-CoV-2 M^{pro} in the presence of different concentrations of tafenoquine or hydroxychloroquine are tested.

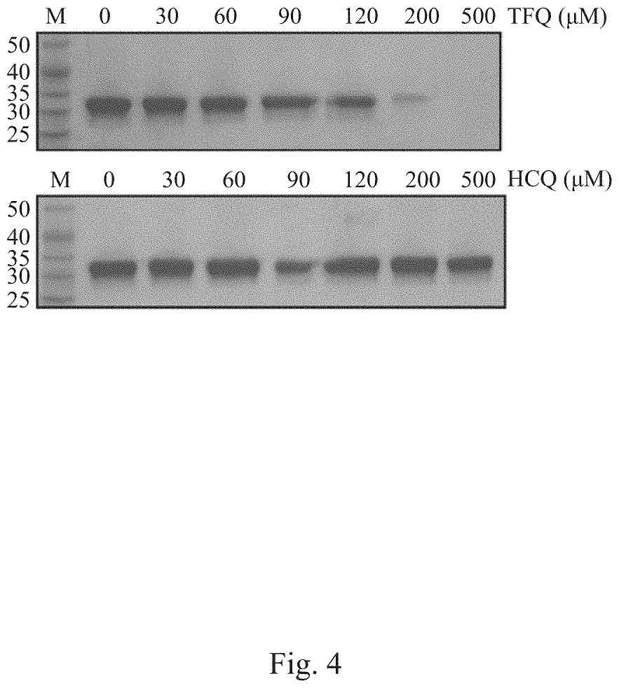

Please refer to FIG. 4, which shows a result of soluble fractions of SARS-CoV-2 M^{pro} in the presence of different concentrations of tafenoquine or hydroxychloroquine. As shown in FIG. 4, the protein bands of SARS-CoV-2 MP′ remain intact at concentrations of tafenoquine up to 90 μM. Further, when the concentration of tafenoquine is above 120 μM, the soluble fraction of SARS-CoV-2 M^{pro} diminishes gradually, suggesting that the conformational change induced by tafenoquine may expose some hydrophobic residues and ultimately result in protein aggregation. However, hydroxychloroquine does not influence the stability of SARS-CoV-2 M^{pro} at concentration up to 500 μM.

To further probe the conformational changes of SARS-CoV-2 M^{pro}, the limited proteolysis assay by trypsin digestion is performed. In the experiment, the protein sample is pre-incubated with tafenoquine in the concentrations of 0, 30, 60 and 90 μM at room temperature for 30 minutes. Proteolysis is performed by mixing SARS-CoV-2 M^{pro} (0.8 mg/ml) with trypsin at a protease-to-protein ratio of 1:10 (w/w) in a reaction buffer (25 mM Tris at pH 8.0, 150 mM NaCl) at 37° C. for 30 minutes. The reaction is stopped by adding SDS sample loading buffer and boiling at 95° C. for 10 minutes and subjected to SDS-PAGE (4%-20%).

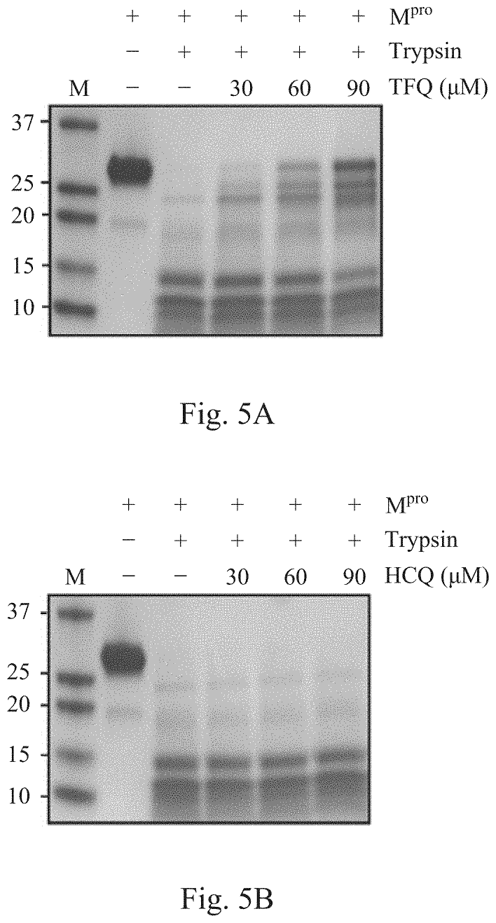

Please refer to FIGS. 5A and 5B, wherein FIG. 5A shows a result of limited proteolysis of SARS-CoV-2 M^{pro} by trypsin in the presence of different concentrations of tafenoquine, and FIG. 5B shows a result of limited proteolysis of SARS-CoV-2 M^{pro} by trypsin in the presence of different concentrations of hydroxychloroquine.

As shown in FIG. 5A, the cleavage pattern of SARS-CoV-2 M^{pro} by trypsin indicates a greater degree of protection of SARS-CoV-2 M^{pro} from trypsin digestion at higher concentrations of tafenoquine. In contrast, as shown in FIG. 5B, no concentrations of hydroxychloroquine tested reduce the cleavage of SARS-CoV-2 M^{pro} by trypsin digestion. Furthermore, results from binding constant measurement by isothermal titration calorimetry (ITC) indicate tafenoquine bound to SARS-CoV-2 M^{pro} with micromolar affinity (Kd=˜10⁻⁵ M). These findings further support the notion that tafenoquine binding induces local conformational changes in M^{pro} that trigger an active-to-inactive form transition, reduce its melting temperature and protease activity, and render it more resistant to trypsin digestion.

<Assessing the Inhibiting Efficiency of Boceprevir or Narlaprevir to SARS-CoV-2 M^{pro}>

Boceprevir (brand name “Victrelis™”) is a first-generation HCV NS3/4A protease inhibitor that was approved by the U.S. FDA for the treatment of chronic hepatitis C (CHC) genotype 1 infection. Narlaprevir (trade name “Arlansa®”) is a second-generation HCV NS3/4A protease inhibitor with improved potency over boceprevir, was first approved by Russian for the treatment of chronic hepatitis C. The structure of boceprevir is shown as Formula (II), and the structure of narlaprevir is shown as Formula (III). In order to assess the inhibiting activity to SARS-CoV-2 M^{pro} of boceprevir and narlaprevir, FRET assay is performed.

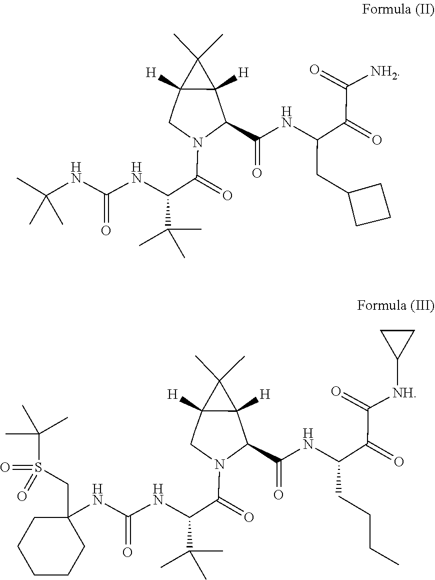 [SMILES: CC(C)(C)NC(=O)N[C@H](C(=O)N1CC[C@H]2[C@@H]([C@H]1C(=O)NC(CC1CCC1)C(=O)C(N)=O)C2(C)C)C(C)(C)C ; CCCC[C@H](NC(=O)[C@@H]1[C@@H]2[C@H](CCN1C(=O)[C@@H](NC(=O)NC1(CS(=O)(=O)C(C)(C)C)CCCCC1)C(C)(C)C)C2(C)C)C(=O)C(=O)NC1CC1]

In the experiment of FRET assay treating with boceprevir or narlaprevir, boceprevir and narlaprevir are respectively pre-incubated at 50 μM along with 1 μM of SARS-CoV-2 M^{pro} for 30 minutes at room temperature in 96-well black optiplate. Then, 20 μM of fluorescent protein substrate is added to each well to initiate the reaction. Substrate cleavage is monitored continuously for 1 hour by detecting mTurquoise2 fluorescence (excitation: 434 nm/emission: 474 nm) using Synergy™ H1 hybrid multi-mode microplate reader. The first 15 minutes of the reaction is used to calculate initial velocity (V₀) by linear regression. The calculated enzyme activity with each compound is normalized to DMSO control. The value of IC₅₀ is calculated by plotting the initial velocity against various concentrations of boceprevir by use of a dose-response curve in Prism 8.0 software.

Furthermore, in the present experiment, other seven FDA-approved anti-HCV drugs, namely telaprevir, simeprevir, grazoprevir, danoprevir, paritaprevir, asunaprevir and HZ1157, are used to react with SARS-CoV-2 M^{pro} so as to further confirm the inhibiting efficiency of boceprevir and narlaprevir to SARS-CoV-2 M^{pro}.

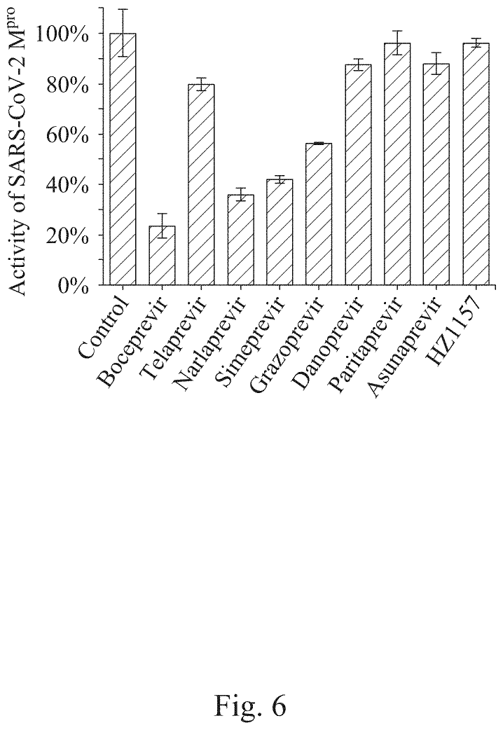

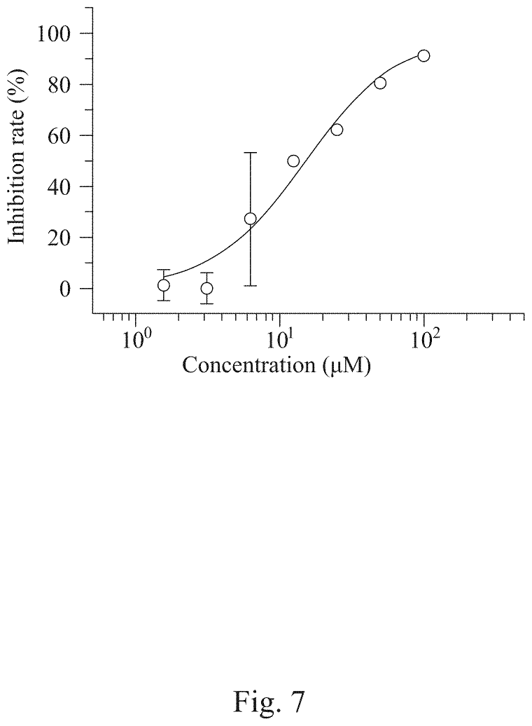

Please refer to FIGS. 6 and 7, wherein FIG. 6 shows a result of relative activity of SARS-CoV-2 M^{pro} after treating with different anti-HCV drugs, and FIG. 7 shows a result of inhibition rate of SARS-CoV-2 M^{pro} after treating with boceprevir. As shown in FIG. 6, compared with other anti-HCV drugs, both of boceprevir and narlaprevir show excellent inhibitory effects on SARS-CoV-2 M^{pro} activity at 76.5±4.8% and 64.1±2.5%, respectively. Furthermore, as shown in FIG. 7, the value of IC₅₀ of boceprevir is estimated as 15.2±3.3 μM. The value of IC₅₀ of narlaprevir is estimated as 40.8±4.6 μM.

Furthermore, in order to further assess the binding effect of SARS-CoV-2 M^{pro} after binding with boceprevir and narlaprevir, the isothermal titration calorimetry (ITC) assay is performed. The experiment of isothermal titration calorimetry is performed on ITC-200 instrument (MicroCal, Northampton, Mass., USA) at 25° C. In detail, SARS-CoV-2 M^{pro} and boceprevir or narlaprevir are dissolved in the assay buffer (20 mM Tris at pH 8.0, 20 mM NaCl, 2% DMSO). By using a syringe, two-microliter aliquots of the boceprevir and narlaprevir (0.75 mM-1.5 mM) in the syringe are respectively injected into cells containing 60 μM-100 μM of SARS-CoV-2 M^{pro} at 3-min intervals. The obtained data are fit to a one-site binding model by using the commercial Origin 7.0 program to obtain ΔH, ΔS, and K_{D} values.

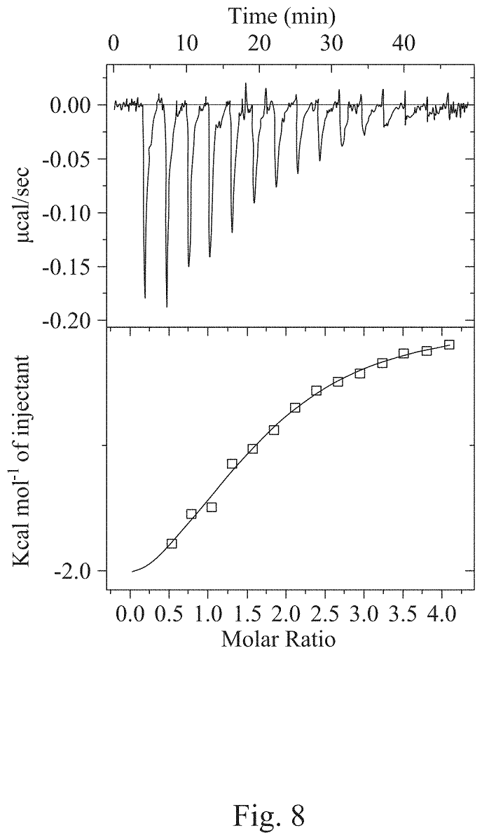

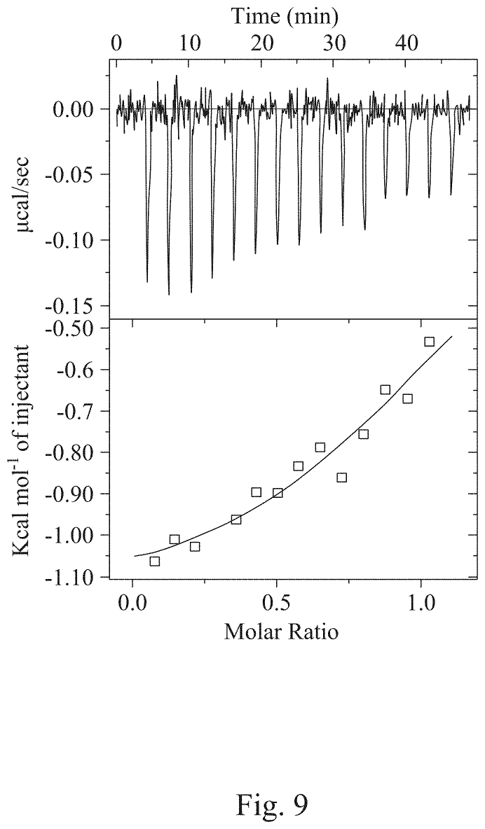

Please refer to FIGS. 8 and 9, wherein FIG. 8 shows a result of isothermal titration calorimetry analysis of boceprevir binding to SARS-CoV-2 M^{pro}, and FIG. 9 shows a result of isothermal titration calorimetry analysis of narlaprevir binding to SARS-CoV-2 M^{pro}. As shown in FIGS. 8 and 9, boceprevir and narlaprevir binding to SARS-CoV-2 M^{pro} are with dissociation constants of 10 μM and 19 μM, respectively. These findings further support the notion that boceprevir and narlaprevir bindings induce local conformational changes in M^{pro} that trigger an active-to-inactive form transition and provide insights in favor of repurposing boceprevir and narlaprevir against COVID-19 by targeting SARS-CoV-2 M^{pro}.

IV. Assessment the Inhibiting Efficiency to the Production of SARS-CoV-2 in Cell Culture System

SARS-CoV-2 (strain NTU02, GenBank: MT066176.1) used in the present disclosure is isolated from a COVID-19 patient at National Taiwan University Hospital and grown in Vero E6 cells. Vero E6 cells are kidney epithelial cells isolated from an African green monkey and commonly used to produce SARS-CoV-2 stocks in many research groups, and in the present experiment, Vero E6 cells are maintained in Dulbecco's modified Eagle's medium (DMEM) containing 10% fetal bovine serum (FBS).

<Assessing the Inhibiting Efficiency of Tafenoquine to the Production of SARS-CoV-2 in Cell Culture System>

The inhibiting efficiency of tafenoquine to SARS-CoV-2 production in cell culture system is assessed by examining the antiviral efficacy of tafenoquine on the viral production and infection rates of SARS-CoV-2 in Vero E6 cells. In the present experiment, Vero E6 cells are seeded in a 24-well plate and subjected to two modes of drug treatment, one in which Vero E6 cells are pre-treated with tafenoquine for 1 hour prior to viral infection (that is, full-time treatment), and the other Vero E6 cells are without tafenoquine pre-treatment (that is, post treatment). In the infection of SARS-CoV-2, Vero E6 cells (1×10⁷) in each well of 24-well plates are washed with PBS, incubated with SARS-CoV-2 diluted in serum-free DMEM containing tosylsulfonyl phenylalanyl chloromethyl ketone (TPCK)-trypsin (2 μg/ml) for 1 hour at 37° C. at a multiplicity of infection (MOI) of 0.001. After infection for 1 hour, the virus inoculum is removed. Then, Vero E6 cells are washed with PBS, and cultured with drug-containing medium until the end of the experiment. After infection and tafenoquine treatment, cell supernatants are collected for further quantification of virus yield on Day 1, Day 2, and Day 3 after infection. The inhibition rate of tafenoquine against SARS-CoV-2 is determined by measuring viral RNA of nucleoprotein using quantitative real-time RT-PCR (qRT-PCR). Furthermore, the viral cytopathic effect (CPE) is observed under microscope and imaged at 3-day post infection.

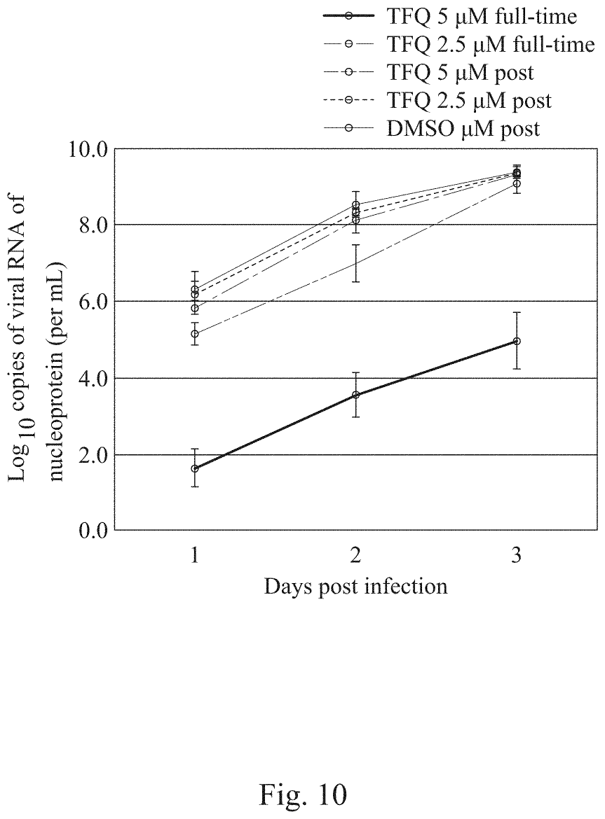

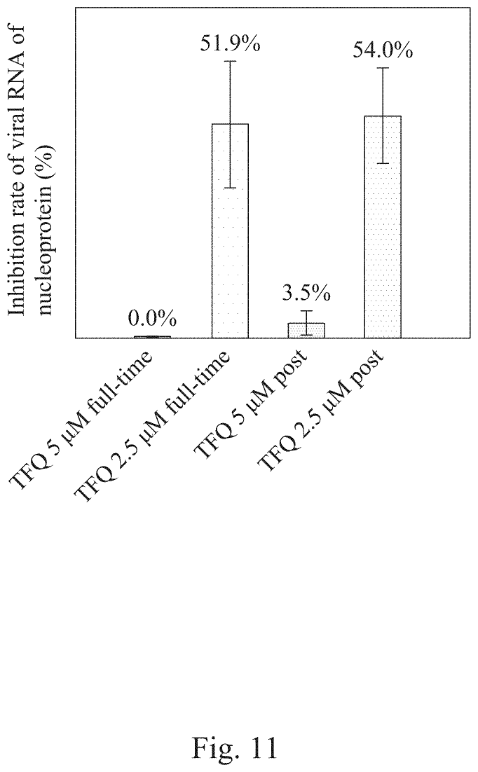

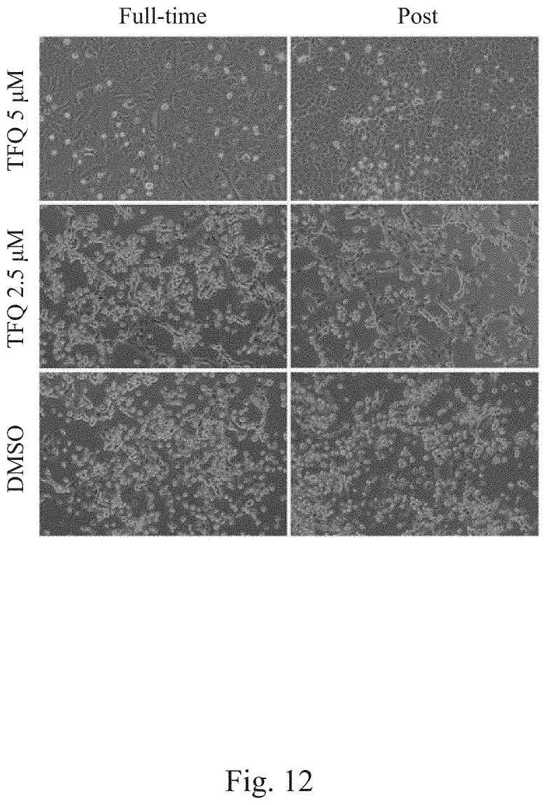

Please refer to FIGS. 10, 11 and 12, wherein FIG. 10 shows a result of amount of viral RNA of nucleoprotein in virus-infected Vero E6 cells which are treated with tafenoquine, FIG. 11 shows a result of the inhibition rate of viral RNA of nucleoprotein on Day 2 in Vero E6 cells treated with tafenoquine with full-time or post treatment, and FIG. 12 shows images of virus-infected Vero E6 cells treated with tafenoquine or DMSO with full-time or post treatment. In FIGS. 10, 11 and 12, the Vero E6 cells post treated with tafenoquine are represented as “post”.

As shown in FIG. 10, tafenoquine significantly represses the yield of viral RNA in cell supernatant on Day 1 to Day 2 after infection, and there is no significant difference of viral RNA between DMSO-treated and tafenoquine (2.5 μM)-treated groups because the former lacked sufficient number of surviving host cells for virus production. Collectively, these data demonstrates that tafenoquine potently reduces SARS-CoV-2 production in the host cells. Furthermore, as shown in FIG. 11, regardless of the treatment method used, the inhibition rate against viral RNA production is approximately 0% to 3.5% and 51.9% to 54% with 5 μM and 2.5 μM tafenoquine, respectively, at 48-hour post infection, implying the half maximal effective concentration (EC₅₀) of tafenoquine is around 2.5 μM.

Furthermore, viral infection can lead to changes in cell morphology and death of host cells, also known as cytopathic effect. As shown in FIG. 12, a significant decrease in SARS-CoV-2-induced cytopathic effect in Vero E6 cells treated with 5 μM tafenoquine treatment compared with the DMSO treatment group is observed, indicating that tafenoquine mitigates cell damages caused by SARS-CoV-2.

<Assessing the Inhibiting Efficiency of Narlaprevir to the Production of SARS-CoV-2 in Cell Culture System>

The inhibiting efficiency of narlaprevir to SARS-CoV-2 production in cell culture system is assessed by examining the antiviral efficacy of narlaprevir on the viral production and infection rates of SARS-CoV-2 in Vero E6 cells. In the present experiment, Vero E6 cells are seeded in a 24-well plate and pre-treated with narlaprevir at various concentrations for 1 hour, and then infected with SARS-CoV-2 at a multiplicity of infection of 0.0003. After infection for 48 hours, the cell supernatants of Vero E6 cells are collected and quantified by measuring viral RNA copies through quantitative real-time RT-PCR. The viral cytopathic effect is also observed under microscope and imaged at 3-day post infection. Furthermore, in the present experiment, the combination of narlaprevir and the first FDA-approved drugs for COVID-19, remdesivir, is also selected to further test the synergistic antiviral effects against SARS-CoV-2.

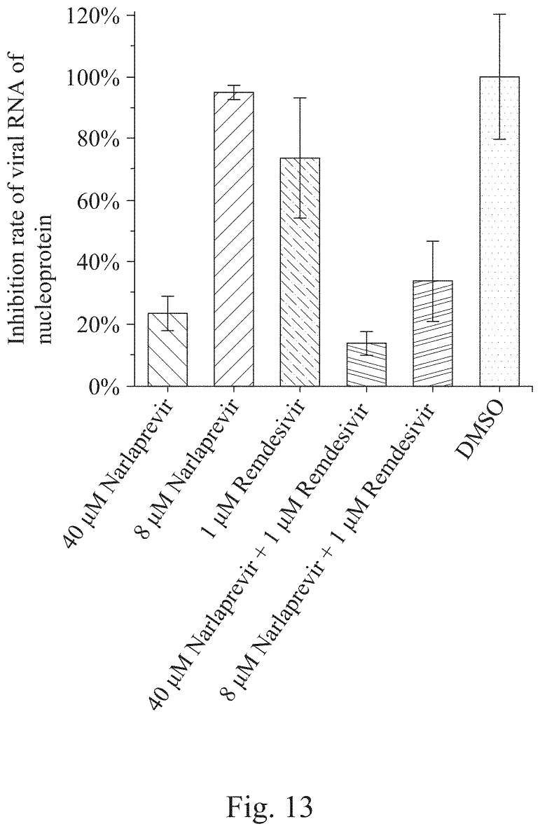

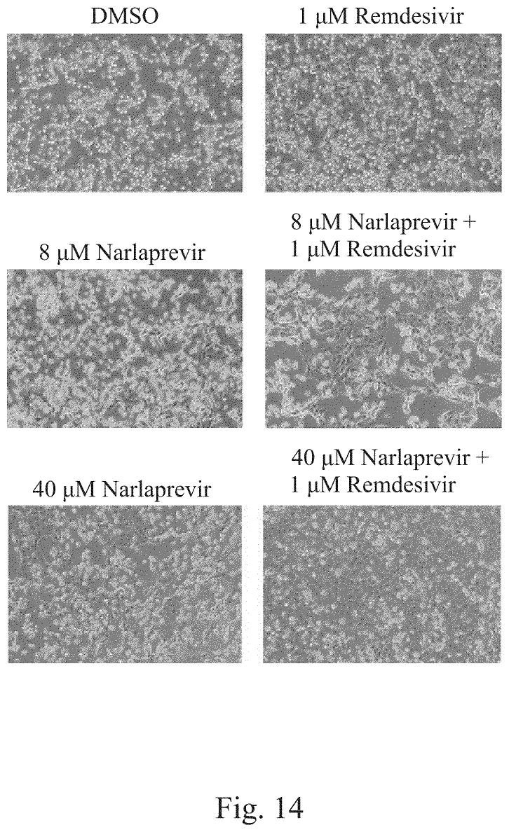

Please refer to FIGS. 13 and 14, wherein FIG. 13 shows a result of amount of viral RNA of nucleoprotein on Day 2 in Vero E6 cells treated with narlaprevir with pre-treatment, and FIG. 14 shows images of virus-infected Vero E6 cells treated with narlaprevir with pre-treatment. As shown in FIG. 13, the inhibition rate against viral RNA production was approximately 76.4% and 26.3% with 40 μM narlaprevir and 1 μM remdesivir, respectively, and the combination of 40 μM narlaprevir and 1 μM remdesivir show synergistic inhibitory effect on SARS-CoV-2 replication with the inhibition rate of 86.2%. Furthermore, the antiviral drug combination with lower dose (8 μM narlaprevir and 1 μM remdesivir) still shows additive effect.

Furthermore, as shown in FIG. 14, the cytopathic effect induced by SARS-CoV-2 infection on narlaprevir and remdesivir pre-treated Vero E6 cells is significantly lesser compared to DMSO control, indicating that narlaprevir and the combinations of narlaprevir and remdesivir at various concentrations mitigate cell damages caused by SARS-CoV-2.

To sum up, all of tafenoquine, boceprevir and narlaprevir have potentials to inhibit the activity of the main protease of a SARS-CoV-2 and then to prevent or inhibit a synthesis of viral RNA of a SARS-CoV-2 in cells, so that tafenoquine, boceprevir and narlaprevir can be used to manufacture a medical composition for use in a treatment of an infection of SARS-CoV-2 so as to treat patients suffering from COVID-19 in order to save lives and reduce the damage provoked by this outbreak.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure covers modifications and variations of this disclosure provided they fall within the scope of the following claims.

What is claimed is:

1. A method for preventing or inhibiting a synthesis of viral RNA of SARS-CoV-2 in a cell, comprising:
contacting the cell with a sufficient concentration of tafenoquine, boceprevir or narlaprevir.

2. The method of claim 1, wherein the sufficient concentration of tafenoquine has a half maximal effective concentration value (EC₅₀) equal to or larger than 2.5 μM.

3. The method of claim 1, wherein the sufficient concentration of boceprevir ranges from 4 μM to 15 μM.

4. The method of claim 1, wherein the sufficient concentration of narlaprevir ranges from 8 μM to 40 μM.

5. The method of claim 1, further comprising:
contacting the cell with a sufficient concentration of remdesivir.

6. A method for inhibiting an activity of a main protease (M^{pro}) of SARS-CoV-2, comprising:
contacting a SARS-CoV-2 with a sufficient concentration of tafenoquine, boceprevir or narlaprevir.

7. The method of claim 6, wherein the sufficient concentration of tafenoquine ranges from 30 μM to 500 μM.

8. The method of claim 6, wherein the sufficient concentration of boceprevir has a half-maximum inhibitory concentration (IC₅₀) of 15.2±3.3 μM.

9. The method of claim 6, wherein the sufficient concentration of narlaprevir has a half-maximum inhibitory concentration (IC₅₀) of 40.8±4.6 μM.

10. A medical composition for use in a treatment of an infection of SARS-CoV-2, comprising:
a therapeutically effective amount of tafenoquine, boceprevir or narlaprevir.

11. The medical composition of claim 10, further comprising:
a therapeutically effective amount of remdesivir.

12. A method for treating a subject suffering from COVID-19, comprising:
administering to the subject in need of the medical composition of claim 1

13. A method for treating a subject suffering from COVID-19, comprising:
administering to the subject in need of the medical composition of claim 1
